(19) United States
(12) Patent Application Publication
(10) Pub. No.: US 2016/0101273 A1
(43) Pub. Date: Apr. 14, 2016

(54) NON-INVASIVE AGENT APPLICATOR

(71) Applicant: MuPharma Pty Ltd, Toorak, Victoria (AU)

(72) Inventors: Harry Unger, Toorak, Victoria (AU); Sean Michael Langelier, St Kilda, Victoria (AU); Mark Unger, Toorak, Victoria (AU)

(73) Assignee: MuPharma Pty Ltd, Toorak, Victoria (AU)

(21) Appl. No.: 14/889,800

(22) PCT Filed: May 6, 2014
(86) PCT No.: PCT/AU2014/050027

§ 371 Date: Nov. 6, 2015

(30) Foreign Application Priority Data
May 6, 2013 (AU) 2013901606

(51) Int. Cl. A61M 37/00

(52) U.S. Cl. CPC A61M 37/0092; A61M 2037/0007

(57) ABSTRACT

An agent carrier delivers an agent to biological tissues by one or more modalities. The modality of delivery may be one or more transportation stimulus that causes the agent to be transported through the agent carrier. The transportation stimulus also enhances or permits penetration of the agent into the tissue. The agent carrier has an agent carrier body that is configured to retain the agent within the agent carrier body. The agent carrier body has a tissue-contacting surface for engaging tissues under treatment. Application of the transportation stimulus causes transportation of the agent through the agent carrier body to the tissue-contacting surface.

### FIELD OF THE INVENTION

The present invention relates to the application of an agent to a target site. In a preferred form, the invention uses ultrasonic energy to transport an agent contained within an agent carrier body having a microstructure formed within it for delivering the agent to the target site.

### BACKGROUND OF THE INVENTION

WO 2007/143796 discloses a method of delivering a molecule and/or particle to a target site using a device that includes generating ultrasound for enhancing the penetration of a molecule and/or particle into the target tissue.

The device of WO 2007/143796 includes an electro-conductive polymeric gel material that is loaded with a molecule and/or particle such as a pharmaceutical or ink etc. Application of an electric field to the electro conductive polymer releases substantially bound molecules or particles within the polymer matrix and, ultimately, such molecules or particles are driven out through the polymer gel by ultrasound to the target tissue surface. At the target tissue surface, penetration of the molecule and/or particle into the tissue is enhanced through sonophoretic mechanisms.

One difficulty relating to this delivery mechanism is that the structure of the polymer gel can degrade over time, for example due to loss of moisture, which results in reduced propagation of the molecule and/or particle by ultrasound. Additionally, gel like polymers are poor transmitters of ultrasound reducing the efficacy of the sonophoretic process. Furthermore, it can be time consuming and non-trivial to properly load an applicator with small volumes of the molecule and/or particle loaded polymeric gel.

In light of these problems, an improved device and mechanism for delivering an agent to a target tissue is sought.

Reference to any prior art in the specification is not, and should not be taken as, an acknowledgment or any form of suggestion that this prior art forms part of the common general knowledge in Australia or any other jurisdiction, or that this prior art could reasonably be expected to be ascertained, understood and regarded as relevant, or combined with other prior art by a person skilled in the art.

### SUMMARY OF THE INVENTION

In one aspect of the invention there is provided an agent carrier for delivery of an agent to biological tissues. Delivery of the agent to the tissues can be by one or more modalities. The modality of delivery can be characterised by a transportation stimulus or stimuli that causes transportation of the agent through the agent carrier. In a preferred form, the transportation stimulus also enhances or permits penetration of the agent into the tissue. Preferred forms of the invention use ultrasonic vibration as the transportation stimulus.

In preferred forms the agent carrier includes an agent carrier body configured to retain agent within the agent carrier body. The agent carrier body has a tissue contacting surface for engaging tissues under treatment, wherein application of the transportation stimulus causes transportation of the agent through the agent carrier body to the tissue contacting surface.

The agent to be delivered can include one or more molecules or particles or one or more molecules and particles in combination. The agent can be a fluid or can be carried in a fluid medium, e.g. by being dissolved, suspended or dispersed in a fluid medium, such as water, oil, an emulsion, a gel or the like. To give but a few examples, the agent can include, proteins, vaccines, nucleic acids, monoclonal antibodies or nanoparticles. In preferred embodiments the agent is a pharmaceutical or pharmaceutical composition. The pharmaceutical or one or more active pharmaceutical components of a pharmaceutical composition may be, without limit, any one of: a synthesised compound; a naturally occurring compound; or a biopharmaceutical. The purpose of the delivery of the pharmaceutical or pharmaceutical composition to the biological tissues can be for any desired clinical reason including: treating, curing or mitigating a disease, condition, or disorder; attenuating, ameliorating, or eliminating one or more symptoms of a particular disease, condition, or disorder; preventing or delaying the onset of one or more a disease, condition, or disorder or a symptom thereof; diagnosing a disease, condition, or disorder, or any agent intended to affect the structure or any function of the body. In other embodiments the agent can be an agent used for cosmetic purposes such as for cleansing, beautifying, promoting attractiveness, or altering the appearance of the body. The agent could also be a marker agent used for creating human or machine perceptible makings, e.g. ink or other. Other types of agents may also be used.

In a an aspect the agent carrier includes an agent carrier body having a tissue contacting surface for engaging tissues under treatment, the agent carrier body including a multiplicity of micro channels extending at least partially through the agent carrier body to the tissue contacting surface enabling transportation of the agent through the agent carrier body to a tissue surface.

The micro channels may also enable agent retention in the carrier body.

Preferably the agent carrier, and most preferably the agent carrier body is able to conduct the transportation stimulus.

The transportation stimulus is the driving force for moving the agent through the agent carrier to the tissue-contacting surface, and may enhance and/or permits the penetration of the agent from the tissue-contacting surface into the tissue.

It is preferred that the transportation stimulus is ultrasound. Ultrasound can enhance and/or permit the transport of agent into the tissue by sonophoresis. The transportation stimulus may be an electrical voltage. Establishing an electric voltage can enhance and/or permit the transport of agent into the tissue via iontophoresis. In some embodiments the transportation stimulus can be both ultrasound and electric voltage used in combination. The ultrasound and electrical voltage can be applied alternately or simultaneously.

In some embodiments the tissue can be any human or animal biological tissue, including mucous membranes and skin. Preferably the tissue is ocular tissue or oral mucosa. In some embodiments the tissue is any plant tissue.

In some embodiments the agent carrier body is made from a semi-conductor material. Preferably the semi-conductor material is silicon. In other embodiments the agent carrier body made from polymer, plastics material, or metal.

In some embodiments the tissue contact surface is flat. In an alternative embodiment the tissue contact surface is contoured to present a convex, concave or toroidal surface to enhance agent delivery to a tissue surface.

In some embodiments an area of contact between the tissue contact surface and the tissue is circular, annular, elliptical, or polygonal.

In some embodiments, the agent carrier body is a unitary structure that includes the tissue contact surface.

In some embodiments the agent carrier body includes a stack of layers including a tissue contact layer, which includes the tissue-contacting surface, and at least one other layer. Preferably, the tissue contact layer has holes extending through the layer to define at least a portion of the micro channels in the agent carrier body. More preferably, a plurality of layers has holes formed therein to enable agent to be transported from one layer to the next. Even more preferably holes formed in one layer of the stack of layers are aligned with holes in an adjacent layer so that a plurality of holes in a plurality of layers cooperate to form the micro channels. Preferably, where a plurality of layers exists, the holes decrease in diameter and increase in number from the first layer to the tissue contact layer. The smaller sized holes in subsequent layers from the first layer to the tissue contact layer can be arranged in clusters so that they align with larger holes in preceding layers.

In some embodiments the agent carrier is able to conduct and/or generate ultrasonic waves.

In some embodiments the agent carrier further includes an agent reservoir for storing agent. The agent carrier may include a plurality of agent reservoirs. The agent reservoir(s) can be formed in the agent carrier body. In some embodiments the agent carrier body can include a reservoir for storing agent. The reservoir can fully or at least partly be formed in the agent carrier body. In some embodiments the reservoir can be at least partly formed external to the agent carrier body. In a layered agent carrier body structure, an agent reservoir may be located in one, or in a plurality of layers in the stack of layers of the agent carrier body. The plurality of agent reservoir(s) within the agent carrier body may have a variety of geometries. In some embodiments the plurality of agent reservoirs within the agent carrier body communicate with each other and with micro channels.

The micro channels and/or agent reservoir(s) are defined by internal exposed surfaces within the agent carrier body, wherein the internal exposed surfaces can be configured to possess predetermined hydrophilic, hydrophobic, and/or electro-conductive properties. At least part of the internal exposed surfaces can be modified or treated to configure their hydrophilic, hydrophobic, and/or electro-conductive properties.

The agent carrier preferably includes a housing configured to mechanically support the agent carrier body in use. The housing can include a mounting arrangement configured to be mounted to an applicator device. The mounting arrangement preferably enables selective attachment and removal of the agent carrier to and from the applicator device, such that the agent carrier can be replaced.

The agent carrier housing also may include a recess or other mounting formation formed therein for receiving the agent carrier body. In some embodiments the agent carrier body can be selectively attached to, or removed from, the recess or mounting formation such that the agent carrier body can be replaced.

The agent carrier can include a port to enable loading of the agent carrier body and/or reservoir(s) with agent.

The agent carrier can further include a stimulus generator, operable to generate a transportation stimulus. The stimulus generator preferably includes an ultrasonic transducer. At least part of the stimulus generator can be formed as part of the agent carrier body.

In a preferred embodiment the agent carrier is a consumable applicator tip adapted for one-time use as part of an applicator device.

In some embodiments the micro channels within the agent carrier have varying cross-sections along their length.

In some embodiments the micro channels within the agent carrier have a variety of different geometries.

In some embodiments, the tissue contact surface of the agent carrier is smooth. In an alternative embodiment the tissue contact surface includes micro-protrusions formed thereon, said protrusions including a cavity defined by at least one of the micro channels.

In a further aspect of the present invention there is provided an agent carrier body for delivery of an agent into a tissue via a transportation stimulus. The agent carrier body includes a tissue contacting surface for engaging tissues under treatment, the agent carrier body including a multiplicity of micro channels extending at least partially through the agent carrier body to the tissue contacting surface enabling transportation of the agent to a tissue surface.

The micro channels can enable retention of the agent within the agent carrier body

The agent carrier body can be adapted to conduct a transmission stimulus to cause or facilitate at least one of the following actions: retention of the agent; transportation of the agent into the agent carrier body; transportation of the agent towards a tissue surface; penetration of the agent into the tissue.

The agent carrier body is preferably made from any one of a semi-conductor material, polymer, plastics material, or metal. In some embodiments the agent carrier body is made from a combination of these.

In some embodiments the tissue-contacting surface can include micro-protrusions formed thereon, said protrusions including a cavity defined by at least one of the micro channels.

In another aspect there is provided an agent body including a tissue contacting surface for engaging tissues under treatment, the tissue contacting surface being at least partly defined by a plurality of protrusions. The protrusions may be in fluid communication with one or more reservoirs forming part of the agent carrier body. Each agent reservoir may comprise a void formed within the agent carrier body. The protrusions may extend outward from an inside of a void and terminate at said tissue contacting surface. The void may be formed by a peripheral structure, where at least part of said peripheral structure may terminate at the tissue contacting surface.

In some embodiments the peripheral structure terminates in a common plane with the protrusions. In others at least some of said protrusions defining the tissue contacting surface extend outward from the void beyond the peripheral structure. In some embodiments, the protrusions may terminate in a plane and the peripheral structure may terminate short of the plane such that the protrusions extend beyond the peripheral structure.

The agent carrier body may further includes a multiplicity of micro channels extending at least partially through the agent carrier body to the tissue contacting surface enabling transportation of the agent to a tissue surface. The micro channels may extend through the agent carrier body to fluidly connect to an agent reservoir.

The agent carrier body of these aspects can include a stack of layers including a tissue-contacting layer, which includes the tissue-contacting surface, and at least one other layer. The tissue-contacting layer preferably has holes extending through it to define at least a portion of the micro channels in the body. In some embodiments a plurality of layers have holes formed therein to enable agent to be transported from one layer to the next. Preferably holes formed in one layer of the plurality of layers are aligned with holes in an adjacent layer so that a plurality of holes in a plurality of layers cooperate to form the micro channels. In some embodiments the holes decrease in diameter and increase in number from the first layer to the tissue-containing layer. The micro channels may have a varying cross-section along their length.

In some embodiments a reservoir for storing agent is at least partly (and optionally fully) formed in the agent carrier body.

The micro channels and/or agent reservoir(s) and/or protrusions are defined by internal exposed surfaces within the agent carrier body. Preferably these internal exposed surfaces are configured to possess predetermined hydrophilic, hydrophobic, and/or electro-conductive properties. In this case, at least part of the internal exposed surfaces could be modified or treated to configure their hydrophilic, hydrophobic, and/or electro-conductive properties.

The agent carrier body may include a port to enable loading of the agent carrier body and/or reservoir(s) with agent.

The agent carrier body can further include a stimulus generator, operable to generate transportation stimulus. The stimulus generator preferably includes an ultrasonic transducer.

In another aspect of the invention there is provided an applicator device comprising an agent carrier and/or an agent carrier body as described herein.

The agent carrier or agent carrier body can be coupled directly or indirectly to a handle unit to facilitate hand held operation of the applicator device. The handle unit preferably includes a mounting arrangement configured to cooperate with a complementary mounting arrangement of the agent carrier and/or agent carrier body.

The handle unit may include an ultrasonic generator to generate ultrasonic waves that are transmitted to the attached agent carrier and/or agent carrier body.

Preferably the agent carrier is a consumable applicator tip adapted for one-time use.

In some forms the agent carrier includes an agent carrier body including a tissue contacting surface for engaging tissues under treatment, the tissue contacting surface being at least partly defined by a plurality of protrusions.

The agent carrier may include one or more agent reservoirs for carrying said agent, wherein said protrusions are in fluid communication with one or more reservoirs forming part of the agent carrier. Each agent reservoir may at partly (or wholly) comprise a void formed within the agent carrier body.

Also disclosed herein is a method of dispensing an agent from an agent carrier. The method comprises holding the agent within an agent carrier, said agent carrier including a solid agent carrier body. The method can further comprise engaging a tissue contacting surface of the agent carrier body with a tissue surface of the biological tissue. The method can further comprise dispensing agent from the agent carrier to the tissue surface by applying at least one transportation stimulus to cause transportation of the agent through the agent carrier body to the tissue surface.

In form embodiments the method further includes applying the transportation stimulus to the tissue via the agent carrier to enhance or permit penetration of the agent into the biological tissue.

Holding the agent within an agent carrier can include holding at least some agent within the carrier body;

In some embodiments the agent carrier body terminates at its tissue contacting surface in a plurality of protrusions. In this case, engaging a tissue contacting surface of the agent carrier body with a tissue surface of the biological tissue, includes engaging the tissue surface of the biological tissue with the protrusions of the agent carrier body.

In another aspect of the invention there is provided a method of dispensing an agent from an agent carrier, an agent carrier body, or an applicator device as described previously, the method including: contacting the tissue-contact surface of the agent carrier with a tissue surface; and dispensing agent from the agent carrier body to the tissue surface and into the target tissue.

In some embodiments of any of the above methods the step of dispensing the agent includes generating ultrasonic waves for agent transport to the tissue contact surface. Even more preferably the method includes propagating ultrasonic waves through the agent carrier to the tissue. This aids the delivery of the agent through the tissues via sonophoresis.

In some embodiments of any of the above methods the step of dispensing the agent can include applying an electrical voltage across the agent carrier body to cause agent transport to the tissue contact surface. The electric voltage can also provide for the transport of agent into and through the tissue via iontophoresis. Even more preferably the method includes propagating an electric current through the agent carrier to the tissue.

In yet another aspect of the present invention there is provided a method of dispensing an agent from an agent carrier, an agent carrier body or an agent applicator device as described herein. The method including, contacting the tissue contacting surface of the agent carrier body with a tissue surface; and dispensing agent from the agent carrier to the tissue surface. The step of dispensing the agent preferably includes generating ultrasonic waves to cause or facilitate agent transportation to the tissue-contacting surface. The method can include the application of ultrasonic waves to the tissue surface to cause or facilitate agent penetration of the agent into and through the tissue via sonophoresis.

The method further includes propagating ultrasonic waves through the agent carrier or agent carrier body to the tissue.

In another aspect the present invention provides a method of loading agent into any one of an agent carrier, agent carrier body, an agent applicator device as described herein.

The method includes, exposing the agent carrier body to the agent to enable filling either of both of, micro channels formed in said agent carrier body or a reservoir in fluid communication with said micro channels, with said agent.

The method can include applying a negative pressure to the agent carrier or agent carrier body to draw agent into the micro channels or agent reservoirs in fluid communication with the micro channels. The method can include applying a positive pressure to the agent carrier or agent carrier body to inject the agent into the micro channels or agent reservoirs in fluid communication with the micro channels.

The step of filling the micro channels or agent reservoirs with the agent can include the application of ultrasonic energy to the agent carrier or agent carrier body to draw agent into the agent carrier or agent carrier body.

In some embodiments, the micro channels in the agent carrier body are loaded by virtue of capillary forces when the agent carrier is in contact with the agent.

As used herein, except where the context requires otherwise, the term “comprise” and variations of such term, such as “comprising”, “comprises” and “comprised”, are not intended to exclude further things, additives, components, integers or steps. Also, as used herein, except where there is express wording to the contrary; specifying anything after the words ‘include’ or ‘for example’ or similar expressions does not limit what else is included.

### BRIEF DESCRIPTION OF THE DRAWINGS

Further aspects of the present invention and further embodiments of the aspects described in the preceding paragraphs will become apparent from the following description, given by way of example and with reference to the accompanying drawings. In the drawings:

FIG. 1A shows a schematic cross-sectional block diagram of an applicator device according to one embodiment, that being applied to a tissue surface and provides an illustration of the overall components of one exemplary applicator device.

FIG. 1B shows a more detailed cross sectional view of the agent carrier body of the embodiment shown in FIG. 1A.

FIG. 1C shows a similar agent carrier body to that of FIG. 1B that includes an ultrasonic transducer.

FIG. 2 provides a cross sectional block diagram of an embodiment of a handle assembly of the applicator device and its basic component parts.

FIG. 3 is a cross sectional view through an agent carrier that takes the form of a single use applicator tip.

FIGS. 4A, 4B, and 4C provide illustrations of various embodiments of a single layer agent carrier body with different micro-channel, and or reservoir arrangements.

FIG. 4D provides an illustration of an embodiment of a first surface and a tissue contact surface of a single layer agent carrier body.

FIGS. 4E, 4F, 4G, and 4H provide illustrations of various embodiments of a multiple layer agent carrier body with different micro-channel and reservoir arrangements.

FIG. 4I provides an illustration of the embodiment shown in FIG. 4H of a first surface and a second surface of a first layer of the agent carrier body, and a first surface and a tissue contact surface of the second layer of the agent carrier body.

FIG. 4J provides illustrations of further example embodiments of agent reservoir contacting layer of an agent carrier body that can store additional agent and replenish the micro-channels as they are depleted of agent during the course of usage.

FIGS. 5A, 5B and 5C provide illustrations of various embodiments of the agent carrier body each of which has a differently configured surface contact layer.

FIG. 5D provides an illustration of two exemplary types of micro-protrusions that extend from the agent carriers shown in FIGS. 5B and 5C.

FIG. 6 provides an illustration of an embodiment of an agent carrier body having a stacked layer arrangement and an agent filling port.

FIGS. 7A and 7B provide illustrations of embodiments of the holes, and the channels defined by the holes, in an agent carrier body that has a stacked layer structure.

FIGS. 7C to 7E provides magnified images of the holes and micro-channels created by the micro-manufacturing process.

FIGS. 8A and 8B are schematic representations of an alternative embodiment of an agent carrier body, and respectively illustrate plan and perspective views thereof.

FIGS. 8C and 8D are schematic representations of an alternative embodiment of an agent carrier body layer having micro channels formed through it, and respectively illustrate plan and perspective views thereof.

FIGS. 8E and 8F are schematic representations of an alternative embodiment of an agent carrier body layer having a reservoir formed therein, and respectively illustrate plan and perspective views thereof.

FIGS. 8G and 8H are schematic representations of an agent carrier body formed by the agent carrier body layer of FIGS. 8E and 8F stacked with the agent carrier body layer of FIGS. 8C and 8D, and respectively illustrate the plan and agent carrier body in unfilled and filled configurations

FIG. 9A and is an electron micrograph of a portion of an agent carrier body of any one of FIGS. 8A to 8H.

FIG. 9B and is an electron micrograph of a single protrusion of an agent carrier body of any one of FIGS. 8A to 8H.

FIG. 10 illustrates a series of four mask designs, each suitable for forming an a respective agent carrier body (or layer thereof).

FIGS. 11A to 11C provide an illustration of a various embodiments in which an agent reservoir is provided in an agent carrier in a location external to the agent carrier body.

FIG. 12A to 12E illustrate steps in various embodiments of charging or recharging methods that can be used in embodiments of the present invention.

FIGS. 13A and 136 illustrate an exploded view and cross sectional view through agent carrier according to one embodiment.

### DETAILED DESCRIPTION OF THE EMBODIMENTS

A preferred form of the present invention will now be described with reference to an exemplary applicator device for delivering an agent to a target tissue site via a transportation modality, which preferably uses ultrasonic waves. The various embodiments of the invention are able to deliver agent through a tissue surface, preferably through ocular tissue, mucous membranes and skin via the application of ultrasonic energy.

The system comprises an applicator device that is preferably hand-held and used for delivering an agent to a target tissue. The preferred form of applicator device includes a handle coupled to an applicator tip. The applicator tip includes an agent carrier body that has micro channels formed in it through which the agent is delivered from within the applicator tip to a target tissue surface. The agent carrier body may be integrated within the applicator tip, or may be a separate component (such as a cartridge) that is attachable to the applicator tip.

The applicator tip may include a reservoir that holds an agent. The reservoir may form part of the agent carrier body, or may be a separate component that is in fluid communication with the agent carrier body.

An ultrasonic transducer forming part of the handle or applicator tip generates ultrasonic energy (waves) which causes the agent to be moved through the micro channels in the agent carrier body, egress through terminal pores of the micro channels at a tissue contacting surface of the agent carrier body and onto the target tissue surface. The ultrasonic waves also enhance and/or permit agent uptake into the target tissue through sonophoresis.

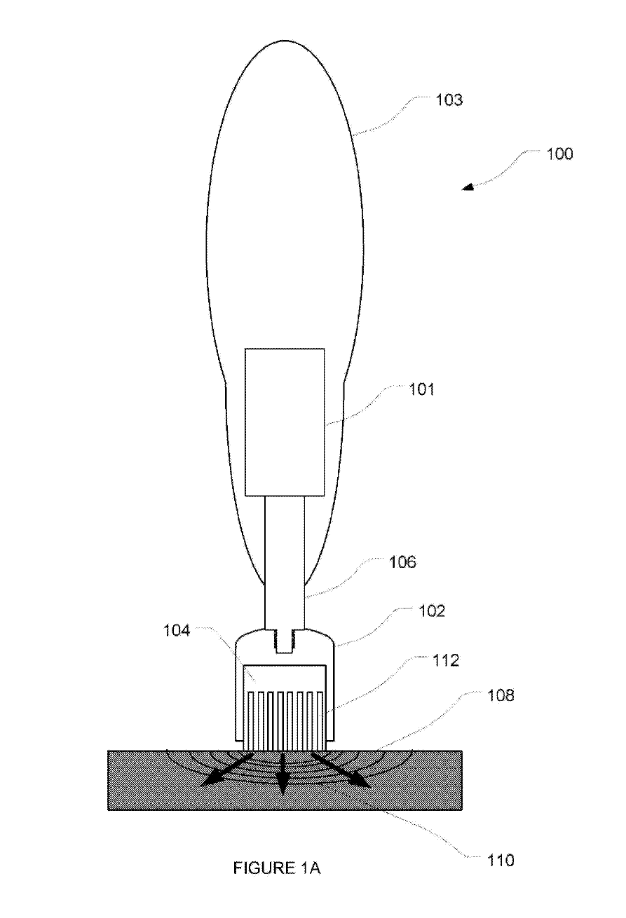

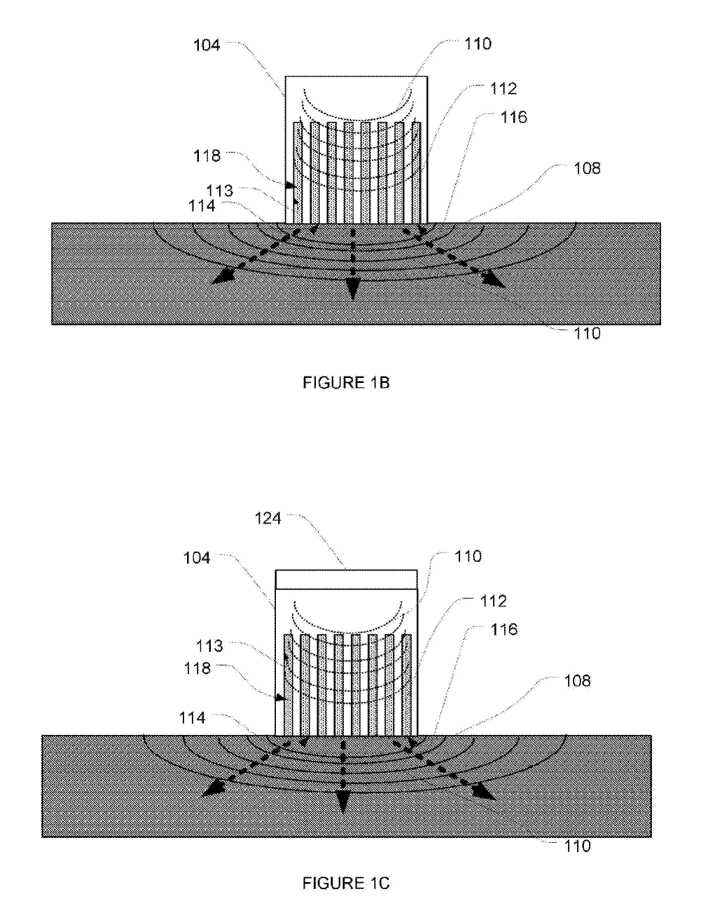

FIG. 1A is a highly schematic diagram illustrating a first embodiment of an applicator device according to the present invention. In this example, an applicator device 100 includes an applicator tip 102 coupled to an applicator handle 103 (entire device not shown). The applicator handle 103 includes an ultrasonic generator 101. The applicator tip 102 is connected to the handle 103 so that ultrasonic energy from the transducer 101 is transmitted to it via a coupling rod 106. The tissue contact surface of the applicator tip 102 is brought into contact with a target tissue surface 108. The ultrasonic generator is then activated, which results in the propagation of ultrasonic waves 110 via the coupling rod 106, through the applicator tip 102 and the agent carrier body 104 and into the target tissue 108. In this embodiment, agent is stored in the agent carrier body 104 and is transported to the target tissue surface 108 via micro channels 112 that have been fabricated within the agent carrier body 104. Ultrasonic waves assist in the transport of agent from the agent carrier body 104 to the target tissue surface 108 via the micro channels 112. Ultrasonic waves also enhance and/or permit the penetration of the agent into the target tissue 108 via sonophoretic effects on tissue ultrastructure.

FIG. 1B provides a more detailed view of the agent carrier body 104 shown in FIG. 1A applied to the tissue surface 108. The agent carrier body 104 has a tissue-contacting surface 114 with micro channels 112 fabricated within the agent carrier body 104 that extend from within the interior of the agent carrier body 104 to the tissue-contacting surface 114. The micro channels 112 terminate as pores 116 at the tissue-contacting surface 114. Agent is provided from the agent carrier body 104, through the channels 112 where it egresses through the pores 116 in the tissue-contacting surface 114, and on to the tissue surface 108. In this embodiment, ultrasound 110 is generated and conducted through the agent carrier body 104. This causes agent 118 stored within the channels 112 to be released from the channels 112 and on to the tissue surface 108. The penetration of agent into the tissue 108 is enhanced and/or permitted by the use of ultrasound, which provides a sonophoretic effect on the tissue.

In the embodiment of FIG. 1A, the applicator handle 103 has an ultrasonic transducer 101, which generates ultrasonic waves 110 that are transmitted through the applicator tip 102 to the agent carrier body 104 via the coupling rod 106. However, in alternative embodiments the applicator tip 102 can be fabricated to include within its structure, a system that is capable of generating ultrasonic waves itself without the need for an external ultrasonic transducer. FIG. 1C illustrates an alternative embodiment in which the agent carrier body 104 additionally includes an ultrasonic transducer 124.

It is preferred that the inner surface(s) of the channel 112 are functionalised. The inner surface 113 of the channels 112 may be functionalised with compounds or molecules having hydrophobic or hydrophilic properties or a combination of both moieties. Alternatively, the surface 113 of the channels 112 may be functionalised by contacting the surface of the channels with small molecules that are adsorbed to the surface of the channels, exposing specific functional groups that have the desired physical and/or chemical properties. The small molecules may be adsorbed through chemisorption or physisorption to the internal surface of the channels. Alternatively, or in addition to changing the water/oil affinity, the inner surfaces of the micro-channels and/or agent reservoirs may be functionalised by enabling them to become electro-conductive.

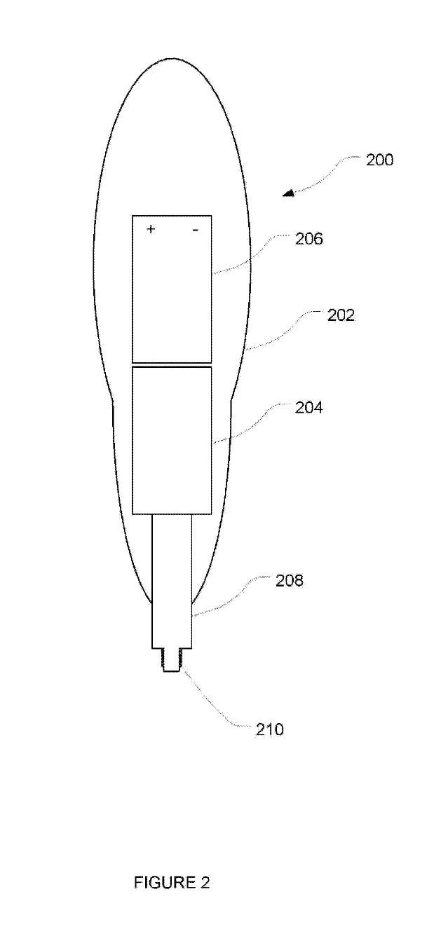

FIG. 2 provides an illustration of an embodiment of the handle assembly 200 of an applicator device. The handle assembly 200 includes a main housing 202, which contains an ultrasonic transducer 204. The transducer is powered by a battery 206 (or alternatively by an external power supply) and is configured to generate ultrasonic waves and transmit them to a coupling rod 208 that terminates in a connector 210. The connector 210 can be of any type for example a screw thread or bayonet fitting or the like, that enables the handle assembly 200 to engage with an agent carrier (through either direct or indirect engagement).

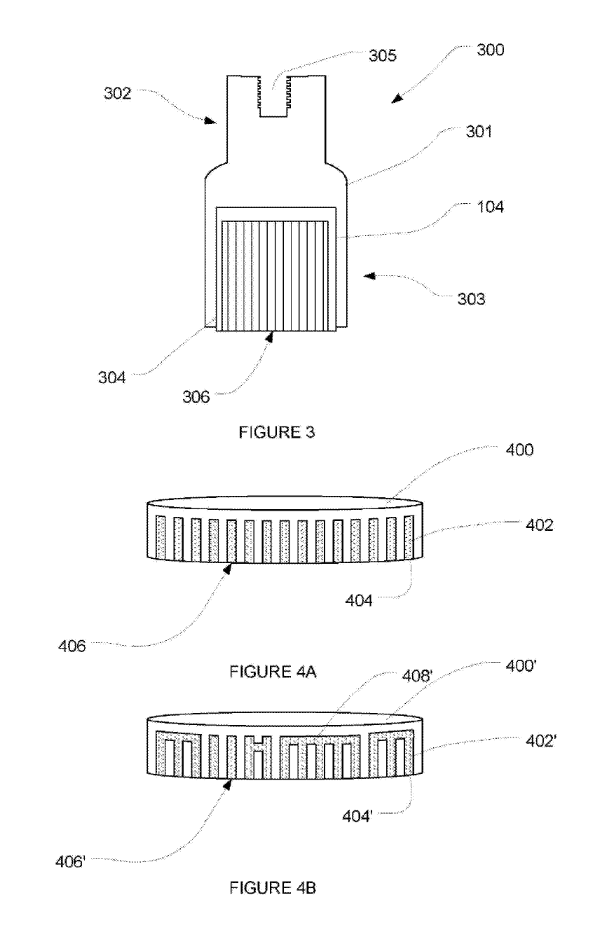

FIG. 3 is a schematic cross section of an applicator tip 300 that may be used with the handle assembly 200 of FIG. 2. The applicator tip 300 includes a housing 301 having a first end 302 and a second end 303. The first end 302 includes a mounting mechanism 305 such as a bayonet fitting or screw thread or the like, that makes a mechanical connection with a connector 210 of the handle assembly 200. The applicator tip 300 further includes a recess 304 at its second end 303 that is arranged to accept the agent carrier body 104. The applicator tip 300 is configured, in use, to carry agent to the tissue-contacting surface 306 of the agent carrier body 104 and deliver it as required to tissue being treated by application of ultrasonic waves. In some embodiments the applicator tip 300 can include an agent reservoir, which is fluidically in contact with the micro channels formed in the agent carrier body 104.

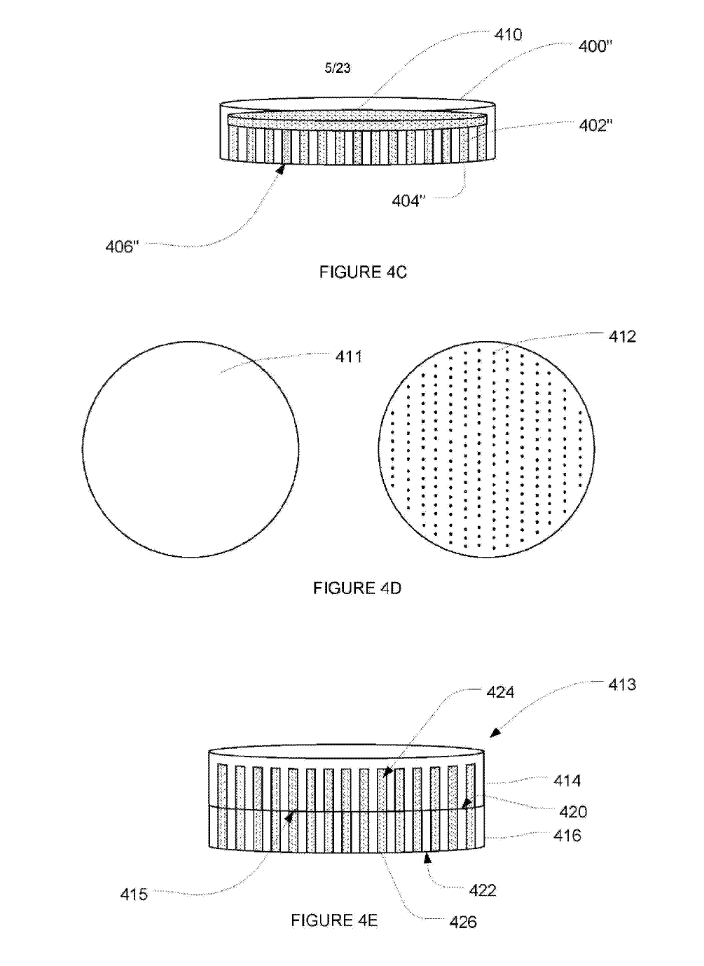

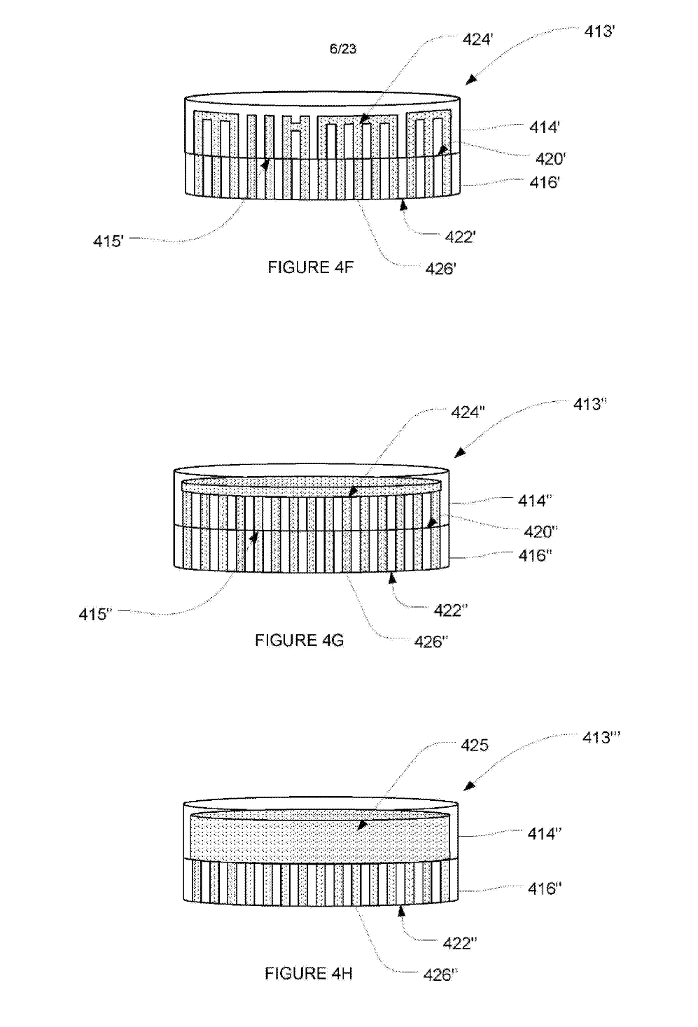

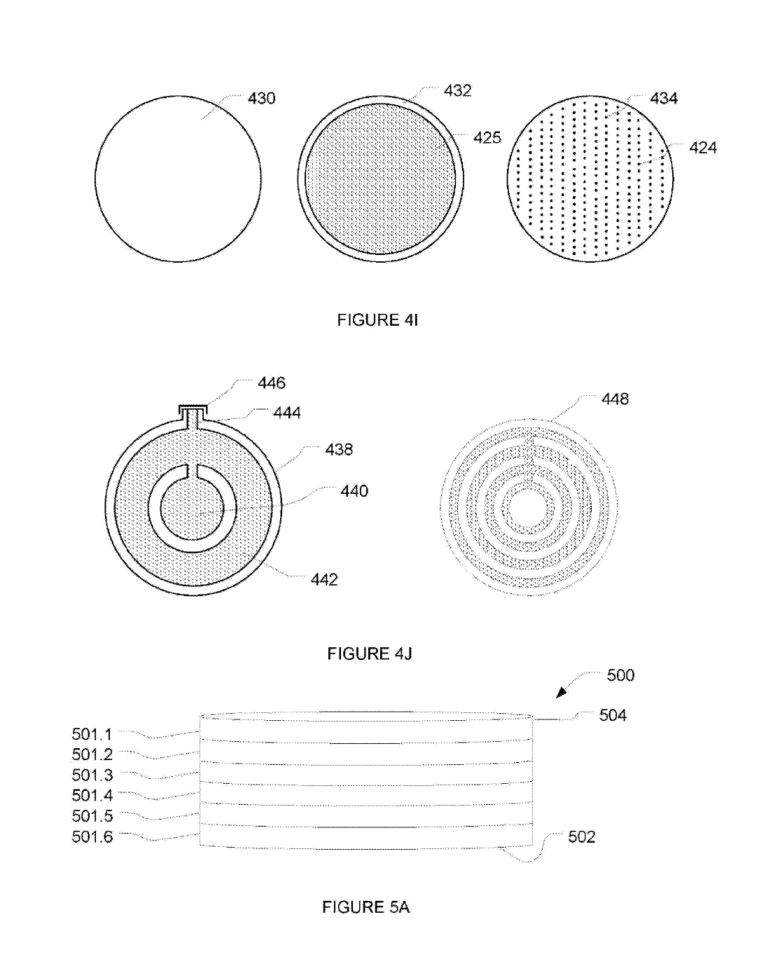

FIGS. 4A, 4B, 4C, and 4D provide illustrations of various embodiments of single layer agent carrier bodies, and FIGS. 4E, 4F, 4G, 4H provide illustrations of various embodiments where an agent carrier body is created from stacked agent carrier layers.

The agent carrier body 400 is formed of a layer(s) of solid material and possesses a number or network of micro channels that may be a variety of geometric shapes and sizes. These micro channels can be used to store or retain an agent and also to deliver agent from within the agent carrier body 400 to a tissue-contacting surface 406 of the agent carrier body 400. The micro channels can be created by a micro-fabrication technique. For instance, in embodiments where the agent carrier body 400 is formed from silicon, the micro channels can be formed by lithography, etching and/or other processes. In embodiments made from metal, plastics or polymers the micro channels can be created by other techniques including the use of lasers of various types and wavelengths and molding and extrusion technologies. The use of these micro-fabrication techniques are particularly desirable as they provide the advantages of retained agent volume accuracy, the benefits of predicable micro-fluidics and further permits refinements such as specialised surface chemical treatment to either or both the exposed tissue-contacting surface and the internal walls lining the micron-scale cavities 402 of the agent carrier body 400. These benefits can be used, for example, to further enhance agent loading, retention and delivery to a target tissue.

The tissue-contacting surface 406 has a series of openings, fenestrations or pores 404. A wide variety of shapes and sizes of pores can be on the order of 10 to 100 μm, but other embodiments may have pore sizes up to 1000 μm. The micro channels 402 extend from the pores 404 in the tissue contact surface 406 at least partially through the agent carrier body 400. The micro channels 402 can be used for both retention of the agent and transportation of the agent to a tissue surface.

The pores 404 may have a patterned appearance and exhibit a range of geometries, for example: close packed hexagon structures, arrayed squares with assorted densities, mixed polygon mosaics, spirals, lines etc. The desired geometries are physically etched into the agent carrier body 400 so as to create arrays of micro channels 402 for retention and/or transport of an agent. The micro channels may be in a variety of shapes for example cylindrical, conical etc.

The walls of the micro channels 402 and/or other internal surfaces within the agent carrier body 400 may be treated such that: they have hydrophilic or hydrophobic characteristics that may be the same or opposite in nature to each other and/or the areas between the pores 404 of the tissue-contacting surface 406. The walls of the micro channels 402 and/or other internal surfaces within the agent carrier body 400 may be treated such that they conduct electric charge or can generate a local electric field that may have the same or opposite polarity to each other and/or the areas between the pores 404 of tissue contacting surface 406.

The agent carrier body 400 can be formed from a unitary piece of material. However, in alternative embodiments the agent carrier body may include a number of layers that are stacked. The use of micro-fabricated solid material as single or multiple layers to create an agent carrier body allows for improved acoustic transmission and thus improved delivery of agent to a target tissue site by ultrasound.

The dimensions and internal lining characteristics of the micro channels 402 and/or other internal surfaces within the agent carrier body 404, and the dimensions and number of layers comprising the agent carrier, will be tailored to suit the agent and the target tissues, and will vary as a consequence of agent properties, dose and formulation requirements, ultrasonic power and heat generation, and the duration of use.

FIG. 4B shows another embodiment, similar to that of FIG. 4A, except that the micro channels 402′ are interconnected by internal linking channels 408. Such a structure provides some level of agent storage in addition to channels 402′ alone.

FIG. 4C represents a further embodiment in which the single layer agent carrier body 400″ has micro-channels 402″ which terminate as pores 404 in the tissue-contacting surface 406″ at one end of the micro-channels 402″, and connect at their other end to an agent reservoir 410.

FIG. 4D provides surface views of a single layer agent carrier body shown in any one of FIGS. 4A to 4C. The agent carrier body 400″ has a first surface 411 and a second surface 412 which is the tissue-contacting surface. As previously discussed, micro-channels extend from within the agent carrier body 400 (from a reservoir 410 or linking channel 408 if present) and terminate as pores 404 in the tissue-contacting surface 412.

In alternative embodiments, the agent carrier body has a stacked layer structure and includes at least two layers. More preferably, one or more layers have additional micro-reservoir volumes formed within them and which are in fluid communication with the micro-channels for holding agent prior to application to the tissues being treated. The micro-reservoir volume may be a single volume or a plurality of small volumes, e.g. each of which is contiguous with one or a group of micro-channels. There may be s single large reservoir volume in the layer furthest from the tissue-contacting layer that is fluidically connected with the channels. Alternatively, there may be multiple micro reservoir volumes, with each of the micro-reservoir volumes being in fluid communication.

FIGS. 4E, 4F, and 4G correspond with FIGS. 4A, 4B, and 4C respectively, except that the agent carrier body 413 includes a first layer 414, 414′, 414″ and second layer 416, 416′, 416″. The first layer 414, 414′, 414″ is as generally described with respect to the single layer embodiment of FIGS. 4A, 4B, and 4C, except instead of having a tissue contacting surface 422, the first layer has an interface surface 415 including pores or blind holes that defines a portion of the micro channels that extend through the first and second layers when the layers are stacked together. The second layer 416, 416′, 416″ includes a first surface 420 that contacts the interface surface 415 of the first layer 414, 414′, 414″ and a tissue-contacting surface 422 having pores 426 that are formed by micro channels 424. As can be seen the micro channels 424 extend from within the first layer, through the second layer 416, 416′, 416″, and terminate at the tissue-contacting surface 422 of the second layer 416, 416′, 416″ as pores 426. In this way, the holes in the first layer 414, 414′. 414″ and second layer 416, 416′, 416″ are aligned to form the micro channels 424 so that the first layer 414, 414′, 414″ and second layer 416, 416′, 416″ are connected permitting fluid continuity in the system.

FIG. 4H illustrates a further alternative embodiment of a double stacked layer agent carrier body 413 in which the first layer 414′″ contains an open-ended agent reservoir 425 that provides agent directly into the micro-channels of the second layer 416″.

FIG. 4I provides surface views of the various layers of a double layered agent 413″ carrier shown in FIG. 4H. The first layer 414″ has a first surface 430 and a second surface 432. The second layer 416″ has a first surface and a second surface (which are the same and are generally represented as 434). The agent reservoir 425, is formed by a recess formed in first layer 414″ that extends partially into it. The second surface 432 of the first layer 414″ is aligned and placed over the interface surface of the second layer 416″ such that substantially all of the micro-channels 424 formed in the second layer are fluidically connected with the agent reservoir 425 in the first layer 414″.

FIG. 4J provides illustrations of further embodiments of agent reservoirs formed in an agent carrier body that can store additional agent and replenish the micro-channels as they are depleted of agent during the course of usage. The reservoirs may connect to micro-channels in the same agent carrier body layer as shown for example in FIG. 4G or connect to micro-channels in a contiguous layer in the agent carrier body as shown for example in FIG. 4H. Agent carrier body 438 includes a reservoir formed by two annular ring shaped reservoir volumes 440 and 442 and includes a conduit 444 extending through a port 446. When a vacuum is applied to the port 446, or the port 446 is injected with agent, a negative pressure or a positive pressure respectively is applied to the reservoir 440, 442. A layer of this type is arranged in a stack of layers to form the agent carrier body, the first layer overlies its adjacent layer such that any holes in the adjacent layer fluidically connect to the reservoir volumes to allow agent to travel via micro channels through the layers and to the tissue-contacting surface.

Agent carrier body 448 is another embodiment in which the reservoir consists of a number of concentric rings each fluidically connected to each other. It will be appreciated that other arrangements of the agent reservoir volumes within a layer are possible without departing from the invention.

Generally, the holes in a lower or intermediate layer of an agent carrier body extend through the whole thickness of that layer and in combination with subsequent fluidically connected holes in other layers, form a micro channel that extends from the tissue-contacting surface in the surface contact layer of the agent carrier. It will be appreciated that in certain instances the holes only extend partway into a particular layer; this can be the case for the first layer as illustrated for example in FIGS. 4E-4G.

As stated previously, it is preferred that the inner surface(s) of the micro channels and other internal surfaces of the agent carrier, such as those of the agent reservoirs, may be functionalised.

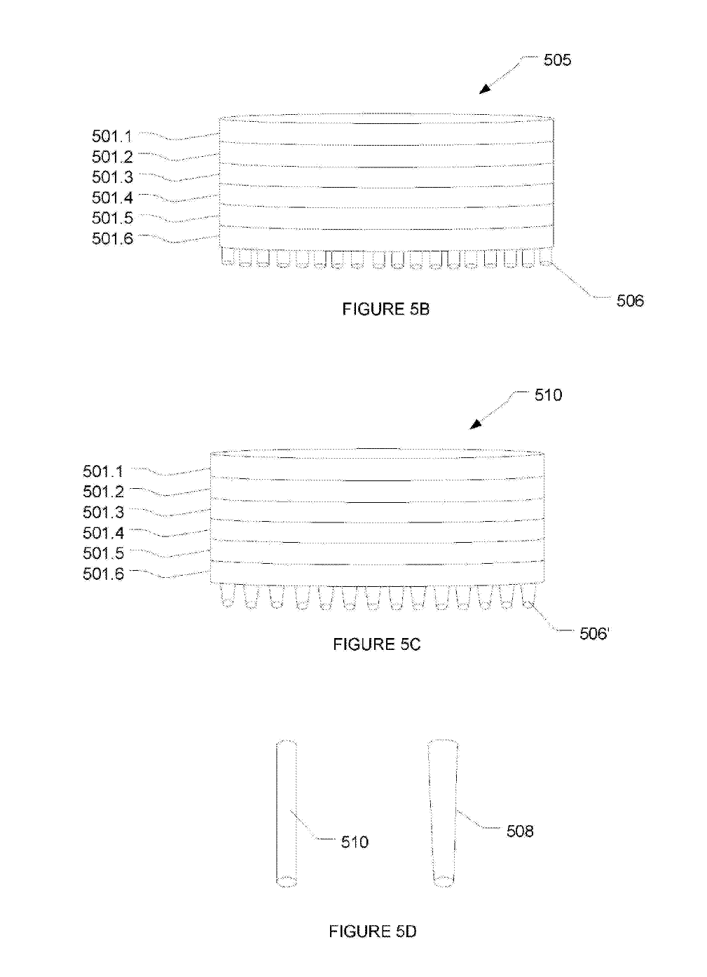

In the embodiments illustrated in FIGS. 5A, 5B and 5C the agent carrier body includes six layers including a surface tissue-contact layer and five layers stacked on top of one another overlying the surface contact layer.

FIG. 5A shows an embodiment of an agent carrier body 500 having six stacked layers 501.1, 501.2, 501.3, 501.4, 501.5, 501.6 The first end of the agent carrier body is a surface tissue-contact surface 502 on layer 501.6 that contacts the tissues being treated. On top of this layer there are a plurality of additional layers and a top most layer 501.1. In this embodiment the agent reservoirs and micro channels for holding and delivering agent (not shown) may extend through some or all of the layers 501.1-501.6 of the agent carrier 500. In some embodiments, the channels extend from the tissue-contact surface 502 in layer 501.6, through intermediate layers 501.5 to 501.2, and terminate in the top-most layer 501.1.

FIG. 5B shows an alternative embodiment of a six stacked layer agent carrier body 505 to that shown in FIG. 5A. In this embodiment, the surface tissue-contact layer 501.6 includes a number of micro-protrusions 506, which in this example are micro-tubules. FIG. 5C shows a further alternative agent carrier body 510 having a similar overall arrangement but in which the micro-protrusions 506′ are micro-needles. The micro-protrusions are hollow, and included channels formed therein that form a part of the system of minor channels for delivering the agent.

Micro protrusions, such as micro-needles and microtubules can be created by secondary fabrication consisting of etching the tissue contact surface 502 of a tissue-contacting layer 501.6 such that the areas between the pores are largely removed. This leaves a wall around each pore of the required protrusion to surround each pore. The micro-needles and microtubules can be of any shape desired. For example, FIG. 50 shows the micro-protrusions as having a cylindrical shape (micro-tubules 510) and other micro-protrusions as having a frustoconical shape (micro-needles 508). In other embodiments, not illustrated, the surface 502 tissue contact layer 501.6 can be provided with other surface treatments, or surface engaging structures, such as a saw tooth structure, ripples, rings or the like to help the agent carrier body interface with the target tissue.

In a preferred embodiment each layer is disc shaped or cylindrical in shape. Preferably the layers have a thickness of from about 0.3 mm to about 1.0 mm, and even more preferably each layer has a thickness of about 0.5 mm. It is preferred that each layer has a diameter of from about 3 mm to about 10 mm, and even more preferably has a diameter of about 5 mm. The thickness dimension and the diameter dimension may vary between layers. While the layers and overall shape of the agent carrier body have been described as being disc shaped or cylindrical in cross sectional shape, as in FIG. 3, other shapes could be employed without departing from the ambit of the invention, e.g. rectangular, square, or other polygon, oval etc. Furthermore, while it is preferred that the overall shape of the agent carrier body is of constant cross section the overall shape of agent carrier body could change along its length e.g. the agent carrier body could be shaped as a frustum (whether conical or otherwise pyramidal), or a prism etc. The overall shape and/or the shape of components of the agent carrier and the agent carrier body can modified in order to maximise the efficiency of the device which is dependent on the transportation modality or combination of transportation modalities employed.

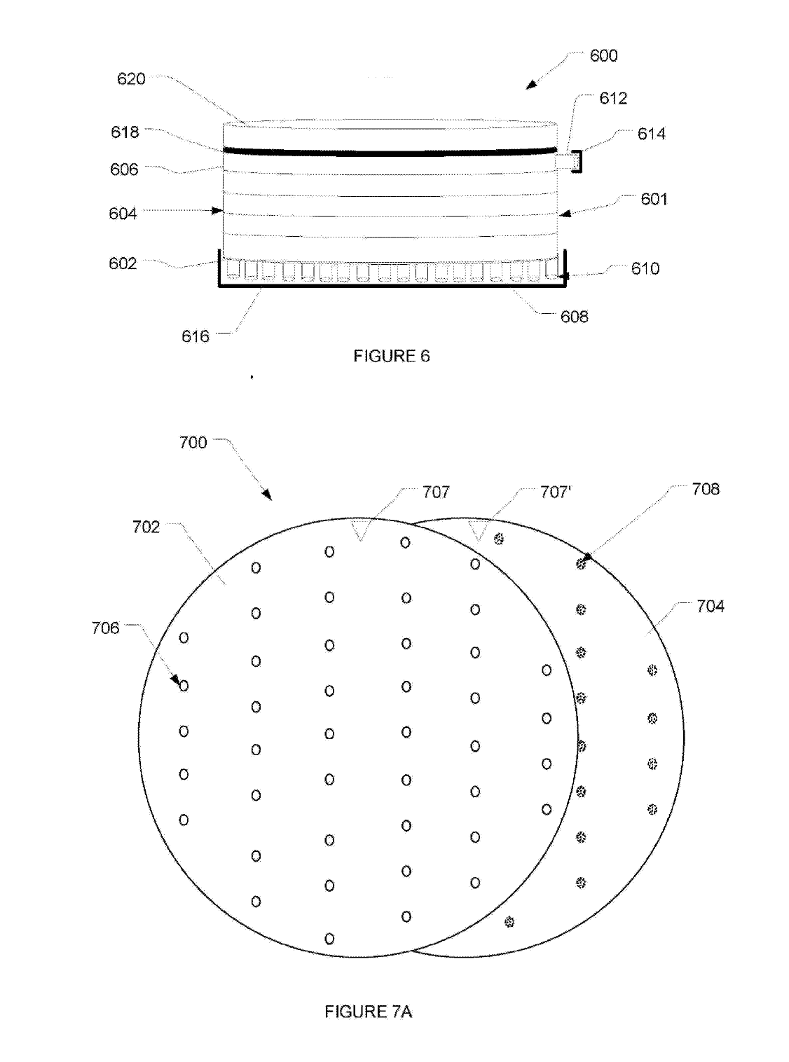

FIG. 6 provides an illustration of an agent carrier 600 having an agent carrier body 601 with stacked arrangement. The stack includes a bottom most layer 602, four intermediate layers 604, a top most layer 606. The bottom most layer 602 has micro-tubules 608 extending to form the tissue-contact surface 610. The agent carrier 600 additionally includes a port 612. In this embodiment the port 612 is part of the first layer 606. The port 612 is connected with micro-channels formed in the agent carrier body 601, preferably via an agent reservoir volume in the first layer 606 so that fluid can flow between them. The port 612 is configured to connect to a vacuum line or pressure injector so that a negative or positive pressure respectively can be applied to the port 612. This allows the agent to be loaded into the agent carrier from an external source. On application of a vacuum to the port 612, agent is drawn through the pores in the microtubules in the tissue contact surface 610, through micro-channels into the stack of layers of the agent carrier body 601 to fill the micro channels and the reservoir volumes. Alternatively, agent can be injected into the agent carrier via the port 612. Using either method, the agent carrier can be charged with an agent.

FIG. 6 also shows a closure or seal 614 applied to the port 612, and a closure or seal 616 applied over the surface contact layer 610. The seal 616 seals the surface of the surface contact layer 610 to maintain sterility and any vacuum that is created within the micro channels. Similarly, seal 614 seals the port 612 for similar purposes. It is preferred that this seal layer is a plastic film.

The embodiment of FIG. 6 also includes an additional layer 618 and an ultrasonic transducer 620. Layer 618 may be a simple insulation layer that serves to cover the fenestrations in the top layer (if the micro-channels extend the entire way through the top layer) to prevent the egress of fluids and/or to prevent release of a contained vacuum.

The transportation modality may use an electric field to cause a charged agent to be transported. The electric field can be provided by applying a voltage to an electrode in the agent carrier using an internal battery in the applicator device or by an external power supply. In a preferred form an electrode is located within the applicator device, a second external electrode, also connected to the applicator device power supply, can be located in such a way that the target tissue effectively becomes an electrode opposite in polarity to that of the internal electrode. The polarity of the electrodes can be selected such that the internal electrode is of the same polarity as the electric charge on the agent. The voltage established between the two electrodes transports an electrically charged agent through the agent carrier to the tissue-contacting surface and can enhance and/or permit the transport of the charged agent into the tissue via iontophoresis. Embodiments of the invention can use multiple delivery modalities using ultrasonic waves and electric current used in combination either alternately or simultaneously. Accordingly, Layer 618 can additionally be modified to include, or alternatively be, a material that serves as an electrode. The electrode can be positively or negatively charged and is used to generate a static or dynamic electric field. In the case where the top surface of the adjacent agent carrier layer does not have pores and the adjacent agent carrier layer is made from a material that is not electro-conductive, there is no direct contact between the electrode and the ions or charged agents contained within the micro channels or reservoirs however, ions and charged agents of the same polarity as that existing on the electrode will be repelled. If the adjacent agent carrier layer is made from a material that is electro-conductive and the adjacent agent carrier layer does not have holes, there is electrical conductivity established with the ions or charged agents contained within the micro channels or reservoirs. This scenario is functionally equivalent to the case where the surface of the adjacent agent carrier layer does have pores (and is not dependent on the electro-conductivity of the adjacent agent carrier layer) and the electrode is in direct contact with the ions or charged agents contained within the micro channels or reservoirs, where a further electrode, opposite in polarity to layer 618 can be placed on, or adjacent to, the target tissue. To complete the electric circuit, the electrode placed on or adjacent to the target tissue may be connected to the agent carrier; applicator handle; or other component of the application device (not shown). An applied voltage can provide the energy required to cause an electrically charged agent of the same polarity as the electrode of layer 618, to flow in the fluid contained in the micro channels of an agent carrier body 601 to migrate through the agent carrier, out of the pores to the tissue surface to be delivered into the tissue by iontophoresis.

This provides an alternative embodiment whereby the agent carrier is able to generate an electric voltage to facilitate the flow of an electric current to transport electrically charged agents through the agent carrier and out of the pores to the tissue.

In some embodiments the agent carrier body includes (as with layer 618), or is itself an electrode to facilitate the transport of a charged agent through the agent carrier and out of the pores to the target tissue. The electrode may be located adjacent to the stack of layers, or may be an electrode layer that is integrated within the stack of layers (as with layer 618).

In the above embodiment, ultrasonic energy and/or electrical voltage provide the energy required to move the agent through the agent carrier to its tissue contact surface where sonophoresis and/or iontophoresis enable the agent to be delivered into the target tissue.

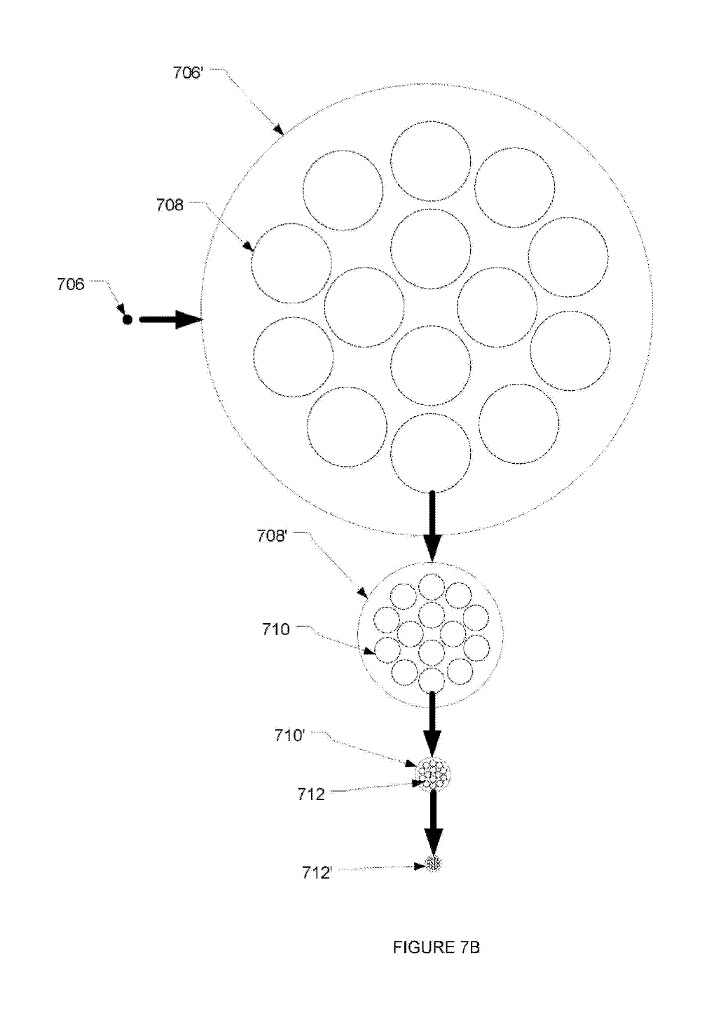

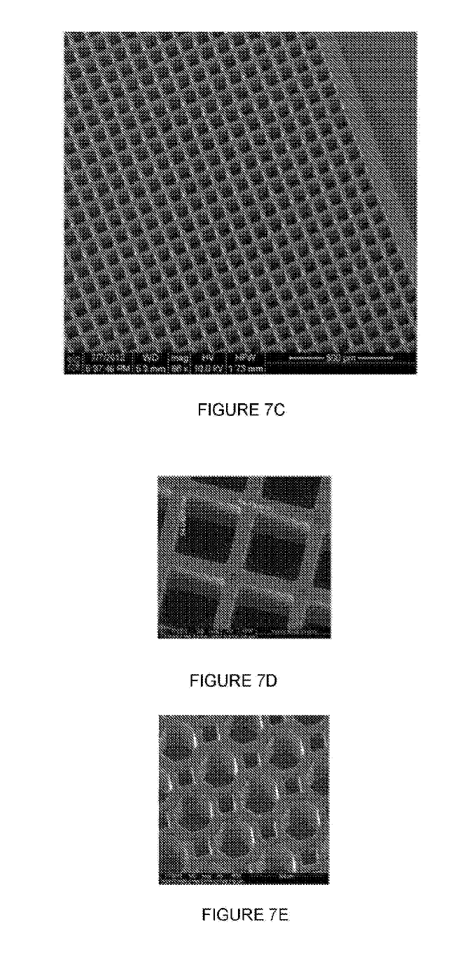

FIGS. 7A and 7B provide an illustration of an embodiment of the holes, and the channels defined by the holes, in a stack of layers forming the agent carrier body according to an embodiment of the present invention. FIG. 7A provides an illustration of a stack of layers 700 that includes two layers, 702 and 704. Layer 702 is a layer that is further from the tissue-contacting surface than layer 704. The layer 702 includes a plurality of holes 706; the layer 704 includes a plurality of holes arranged as a cluster of holes 708. These layers 704, 702 are arranged adjacent to each other in the stack of layers 700 such that each cluster of holes 708 in layer 704 is aligned with a hole 706 in layer 702. The holes in the layer 704 are more numerous and smaller than the holes in layer 702. To facilitate alignment in the layers during device fabrication each layer 702, 704 can be provided with a datum point or structure 707, 707′ which define the alignment of the layer. Layers can then be aligned with their respective datum points 707, 707′ arranged in a predetermined fashion (e.g. aligned with each other) to achieve correct alignment of holes in respective layers 702, 704, thereby forming micro channels that extend through multiple layers of a stack 700.

FIG. 7B provides a further illustration of the variation and alignment between holes of different sizes in different stack layers of the agent carrier body. Hole 706′ is a magnified version of hole 706. The hole 706′ overlies a first cluster of holes 708 (shown in dotted lines) in the next adjacent stack layer. Hole 708′ is a magnified version of hole 708. The hole 708′ overlies a corresponding cluster of holes 710 (shown in dotted lines) in the next adjacent stack layer. Similarly Hole 710′ is a magnified version of hole 710. The hole 710′ overlies a corresponding cluster of holes 712 (shown in dotted lines) in the next adjacent stack layer. Hole 712′ is a magnified version of hole 712 and so on until the final layer.

Multiple layers can be arranged such that progressing from the top most layer, through the intermediate layers, to the surface contact layer, the diameter of the holes decreases and the number of holes may be increased. Each subsequent layer includes a cluster of holes that is in alignment with a hole in the adjacent subsequent layer. For example, a first layer (which may be the top most layer or an upper one of the intermediate layers) has a number of holes. This first layer overlies a second layer, wherein the second layer has clusters of holes that are arranged beneath the holes in the first layer. This second layer may overlie a third layer and each hole in each of the cluster of holes in the second layer overlies a further cluster of smaller holes in the third layer (additional layers may also be provided in this manner).

The channels define a flow path for the agent through the agent carrier body to the tissue surface. The channels are defined initially by the diameter of the holes in the first hole possessing layer. Subsequent layers have clusters of holes that are aligned with the holes in this first hole possessing layer. Therefore, progressing from the first hole possessing layer through subsequent layers, the channels become multi-furcated into numerous branches. It will be understood that these numerous branches all form a part of the channel.

FIGS. 7C, 7D, and 7E show magnified images detailing examples of micro-channels created by a micro-manufacturing process. FIGS. 7C and 7D (7D showing a higher magnification of 7C) shows a layer in which the holes have square cross-sections. FIG. 7E shows a layer that includes holes having square and hexagonal cross-sections.

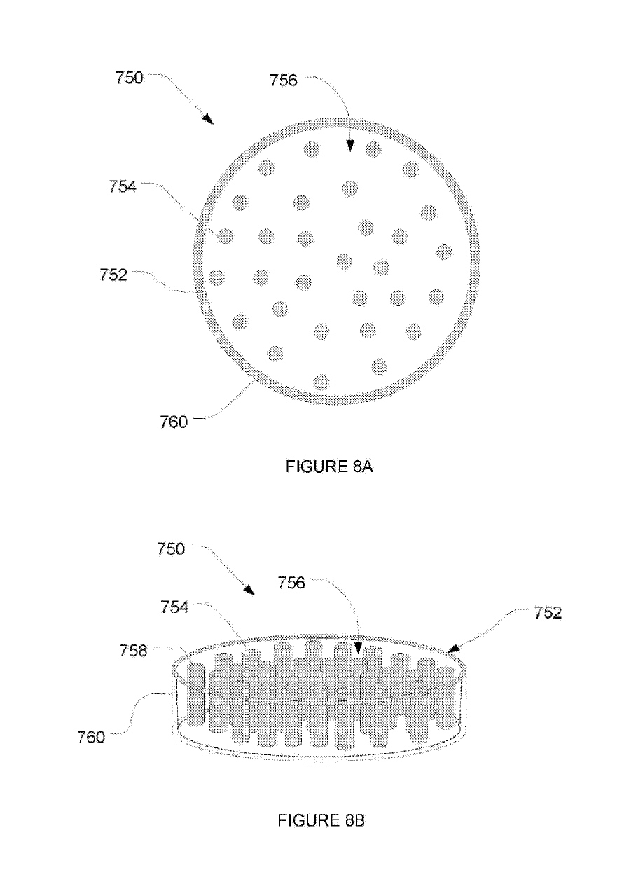

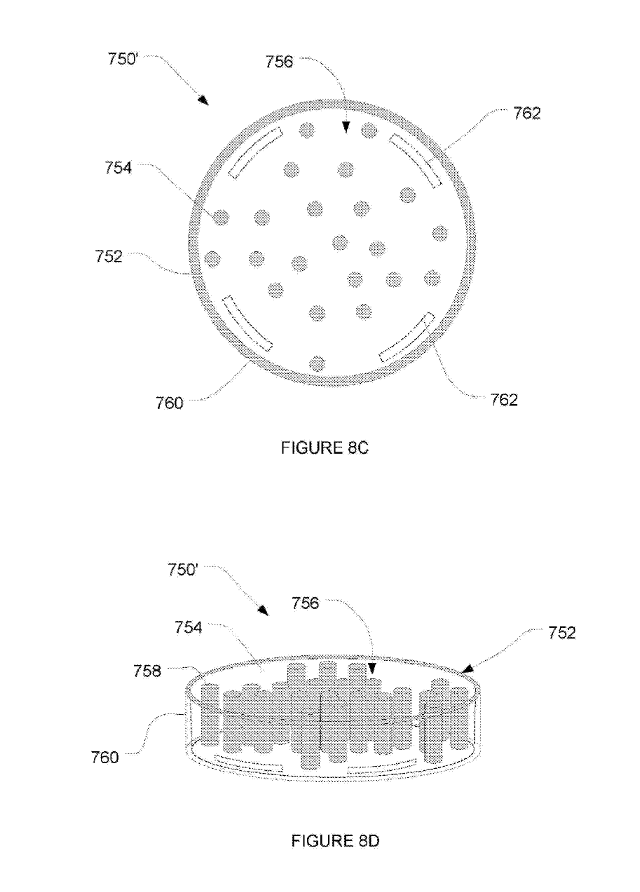

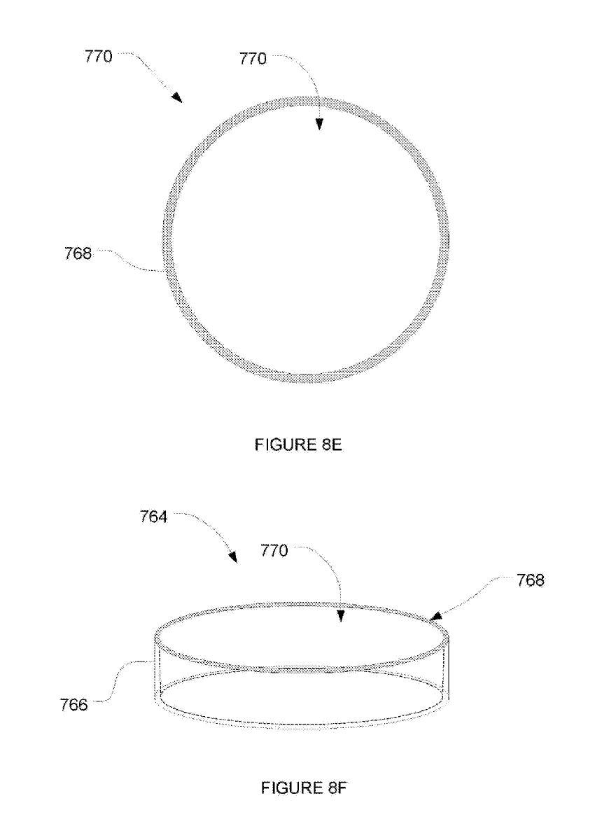

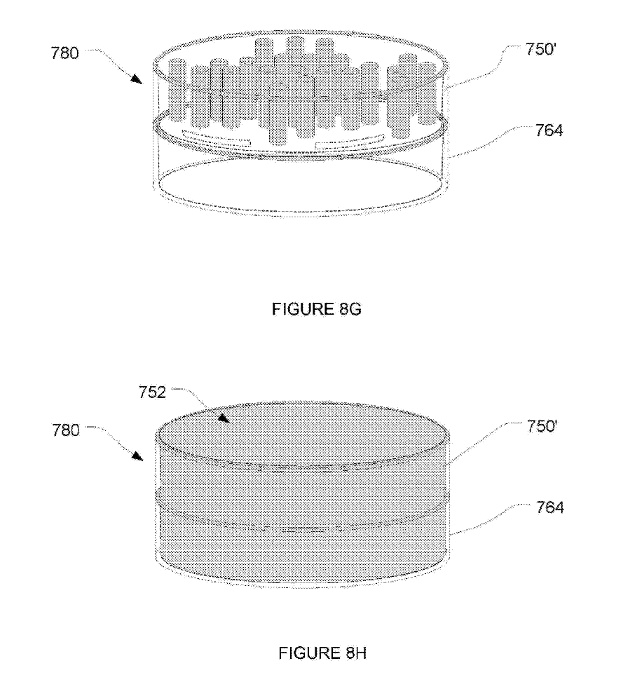

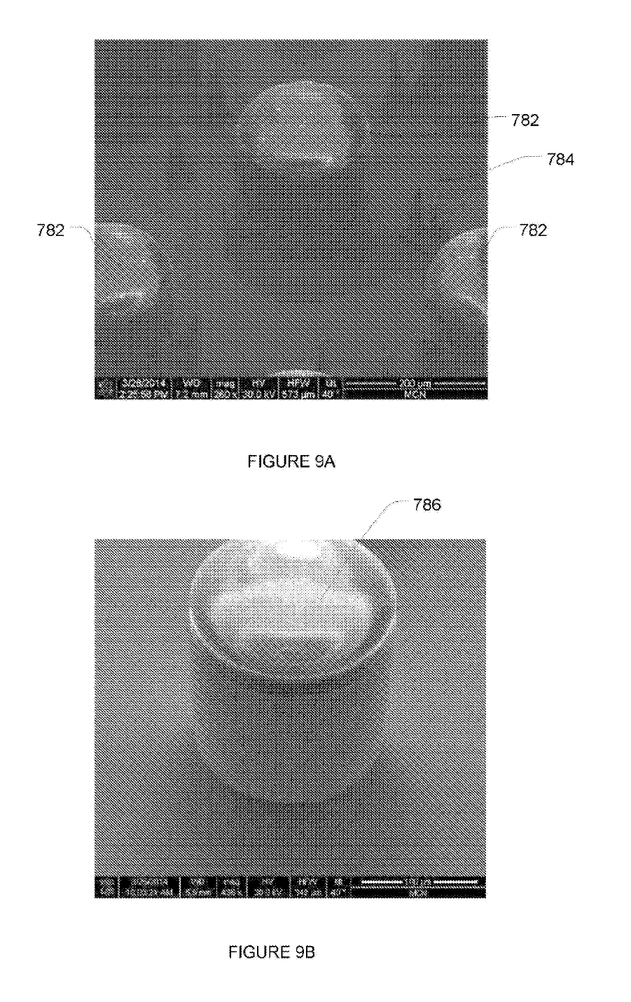

FIGS. 8A to 8G illustrate schematic representations of alternative embodiments of an agent carrier body and agent carrier body layers.

In this embodiment the agent carrier body 750, can be used for delivery of an agent to a tissue via a transportation stimulus. The agent carrier body 750 includes a tissue contacting surface 752 for engaging tissues under treatment. In this example the tissue contacting surface is defined, at least partly by a plurality of protrusions 754.

The protrusions 754 may be of any shape, but in the present example are generally cylindrical. Preferably the protrusions have a constant cross sectional shape along their height. The protrusions 754 extend outward from an inside of a void 756 that is formed within the agent carrier body 750. The outward ends 758 at least partly define the define the tissue contacting surface of the agent carrier body 750.

The void 756 is formed by a peripheral structure 760, which in this case takes the form or a peripheral wall or rim. The rim 760 also defines part of the tissue contacting surface 752.

The peripheral structure 760 in this embodiment terminates in a common plane with the protrusions, to define a planar tissue contacting surface 752. However, in other embodiments the at least some of said protrusions 754 can extend beyond, and/or stop short of the peripheral structure so that tissue contacting surface 752 is not planar. In some embodiments the protrusions 754 may all extend beyond the peripheral structure 760.

The void 754 acts as a reservoir to hold agent within the agent carrier body 750. However unlike previous embodiments this reservoir is located on the tissue contacting surface side of the agent carrier body.

The protrusions 754 are located within the reservoir so that they are in fluid communication with the agent in the reservoir. This allows the protrusions 754 to act on the agent within the agent carrier body 750 and transmit the transportation stimulus into the agent, whereas in the embodiments above the walls of the micro channels acted on the agent within the agent carrier body.

Embodiments of this type generally have more volume for holding agent than embodiments described above. By having a larger filling volume, the possibility of air entrapment may also be reduced. These improved filling properties may give certain embodiments improved filling accuracy and repeatability, which contributes to an increase in dose accuracy, that may be important in medical applications. Furthermore the improved filling may lead to better ultrasonic energy transmission as dampening by retained air spaces is reduced.

It is preferred that the inner surface(s) of the void 754 are functionalised. The inner surface of the void 754 and the protrusions 752 may be functionalised with compounds or molecules having hydrophobic or hydrophilic properties or a combination of both moieties. Alternatively, the surface of the void 754 and the protrusions 752 may be functionalised by contacting the surface of the channels with small molecules that are adsorbed to the surface of the channels, exposing specific functional groups that have the desired physical and/or chemical properties. The small molecules may be adsorbed through chemisorption or physisorption to the internal surface of the channels. Alternatively, or in addition to changing the water/oil affinity, the inner surfaces of the micro-channels and/or agent reservoirs may be functionalised by enabling them to become electro-conductive. In a preferred form loading of the agent carrier body is performed by virtue of capillary forces when the agent carrier is in contact with the agent.

FIGS. 8C and 8D show an agent carrier body layer 750′. In general the agent carrier body layer 750′ is the same as the agent carrier body 750 and like features are like numbered. However the agent carrier body layer 750′ additionally includes one or more micro channels 762 extending through it. The micro channels 762 extend through the agent carrier body layer so that the reservoir 756 may be fluidly connected to an adjacent agent carrier body layer as in previous embodiments. In this example, four micro channels are used.

FIGS. 8E and 8F are schematic representations of an agent carrier body layer having a reservoir formed therein. The agent carrier body layer 764 is generally cylindrical in form and includes a peripheral wall 766 that defines a reservoir volume 770 within it. In use the agent carrier body layer 764 is stacked on the agent carrier body layer 750′ such that the outer rim 768 of the wall 766 contacts the back of the agent carrier body layer 750 such that a reservoir volume 770 closed. The micro channels 762 in the agent carrier body layer 750′ allow agent within the reservoir volume 770 to pass into the reservoir 756 for dispensing.

FIGS. 8G and 8H are schematic representations of an agent carrier body formed by the agent carrier body layer of FIGS. 8E and 8F stacked with the agent carrier body layer of FIGS. 8C and 8D to form an agent carrier body 780. The agent carrier body 780 includes a stack of layers including the tissue-contacting layer 750′ which includes the tissue contacting surface 752 and one other layer 764. More layers could also be stacked to form an agent carrier body.

In FIG. 8H the agent carrier body 780 is shown filled with agent. In this configuration the agent is filled to the tissue contacting surface 752.

FIG. 9A and is an electron micrograph showing a portion of an agent carrier body (or layer thereof) of the type schematically illustrated in FIGS. 8A to 8H. FIG. 9A shows part of three pillars 782 that operate as protrusions 754. The surface 784 is the base of the void 756 from which the pillars 782 extend. FIG. 9B and is an electron micrograph showing a close up portion of another pillar 786. As can be seen these embodiments from their respective scales, the pillars 782 and 786 are around 200 micrometres wide and a similar height. However in other embodiments different heights and widths may be used.

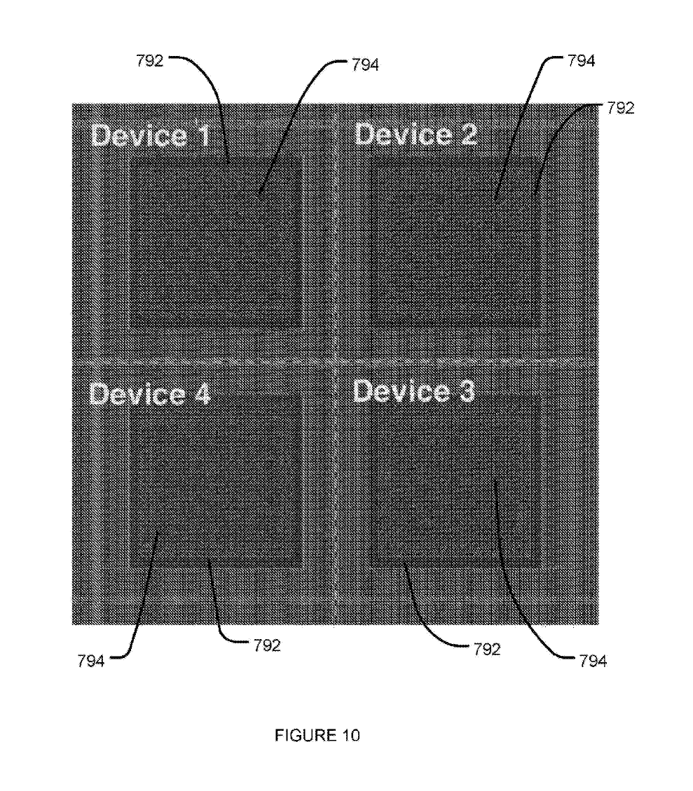

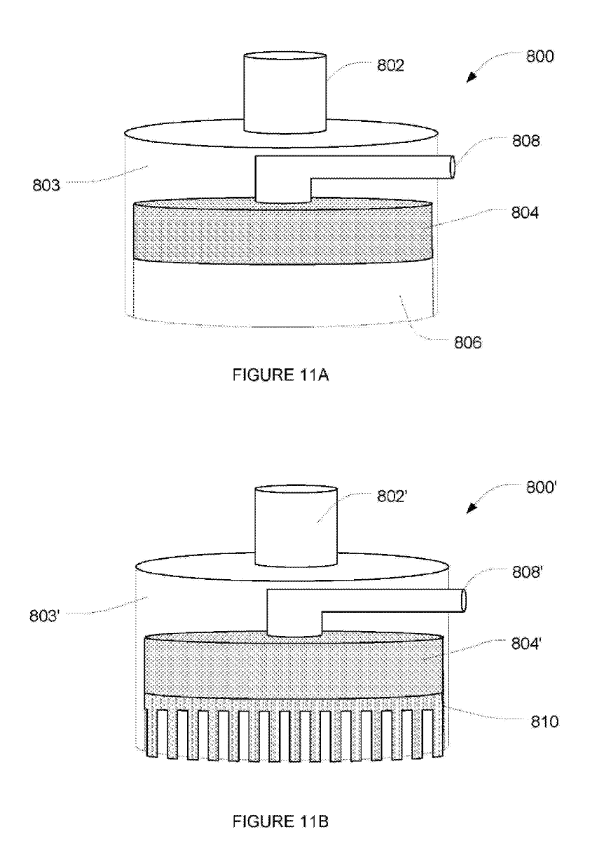

FIG. 10 illustrates a series four mask designs, each suitable for forming an a respective agent carrier body (or layer thereof). The masks are used in a micromachining process for forming the protrusions and peripheral structure of a tissue contacting surface of an agent carrier. The protrusions are to be arranged in a pattern, In this example in a regular array.

In FIG. 10 the mask for each device (Devices 1 to 4) includes a first mask section 792 for defining a square peripheral wall. Device 1 includes an array of 25 mask sections 794 arranged in a 5×5 array to create a 5×5 array of protrusions. Device 2 includes an array of 16 mask sections 794 arranged in a 4×4 array to create a 4×4 array of protrusions. Device 3 includes an array of 9 mask sections 794 arranged in a 3×3 array to create a 3×3 array of protrusions. Device 4 also includes an array of 16 mask sections 794 arranged in a 4×4 array to create a 4×4 array of protrusions. As can be seen, the protrusions of Device 4 are spaced more widely than that of Device 2.

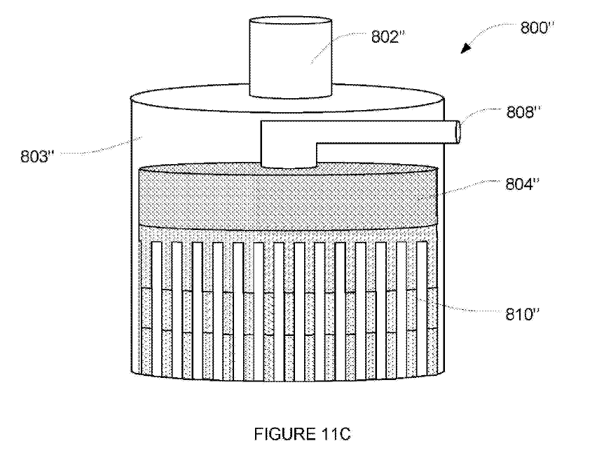

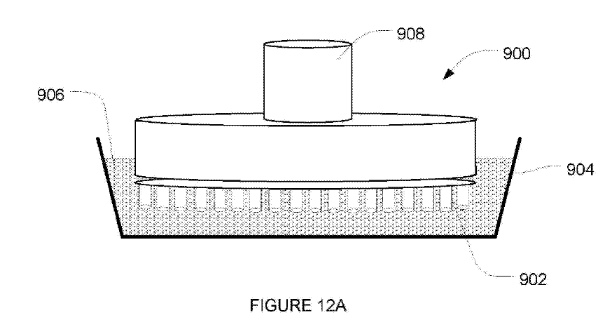

FIGS. 11A, 11B, and 11C illustrate an embodiment in which the agent reservoir is provided within the agent carrier as a separate component to the agent carrier body.

FIG. 11A illustrates a portion of an applicator according to a further embodiment of the present invention. In this figure there is illustrated an embodiment of an applicator tip 800 attached to a coupling rod 802, for coupling the applicator tip 800 to a handle portion of a hand-held agent applicator device. The applicator tip 800 includes an agent reservoir 804 formed within the tip's housing 803. The housing 803 also includes a recess area 806 for receiving an agent carrier body. The agent reservoir 804 includes a port 808. The port 808 may be configured for a number of different uses. In certain embodiments the port 808 may be used to inject the agent reservoir 804 with an agent. In other embodiments the port 808 may be used to apply a vacuum to the agent reservoir 804 to draw agent into the reservoir 804.

FIG. 11B provides applicator tip 800′ with an agent carrier body 810 inserted into the recess area 806 (not shown due to the presence of the agent carrier body 810). As will be appreciated from the description in FIG. 11A, the agent reservoir 804 may be filled with an agent by suction applied to the port 808′ whereby the agent is drawn through the agent carrier body 810 via its micro channels for storage/holding in the reservoir 804. Alternatively, port 808′ may be used to directly inject the agent reservoir 804′ with an agent which then fills both the reservoir 804′ and the micro channels in the agent carrier 810 with the agent.

FIG. 11C provides a further embodiment of an applicator tip 800″ as generally described above, and accordingly corresponding features have been like numbered with the addition of double prime to indicate the change of embodiment. The applicator tip 800″ is connected to coupling rod 802′. It includes an agent reservoir 804″ and a stacked agent carrier body 810″. In other respects it is the same as the previous examples.

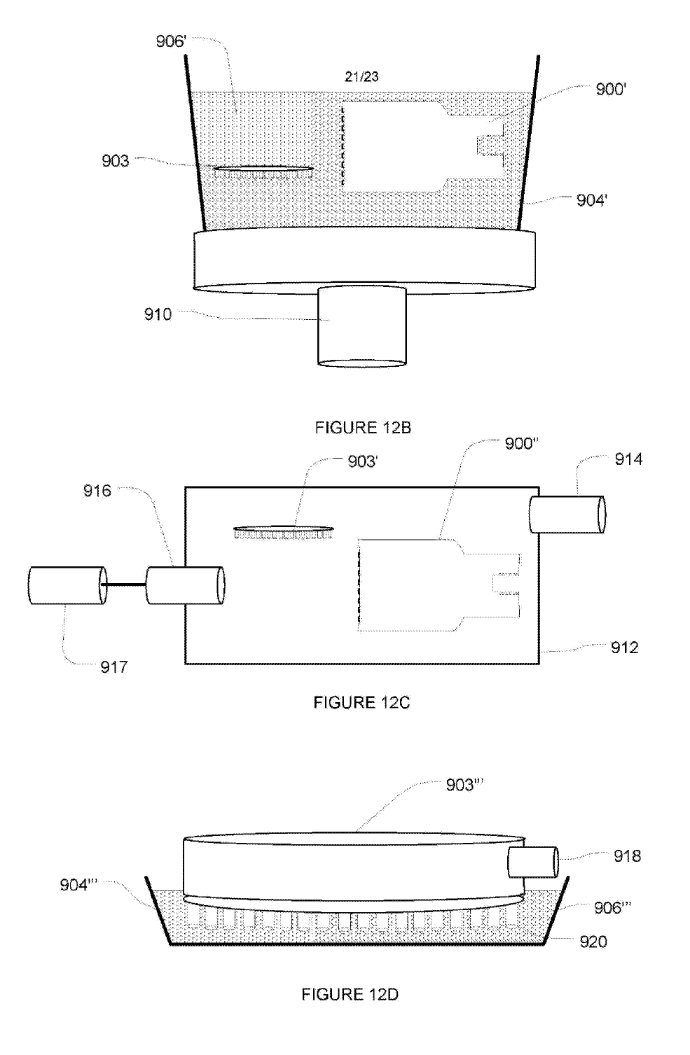

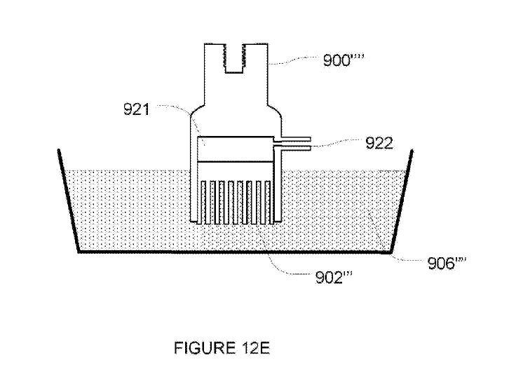

FIGS. 12A, 12B, 12C, 12D, and 12E provide illustrations of mechanisms, modifications and methods of charging an agent carrier with agent and/or other substances that assist in the loading, retention and delivery of agent by the system.

The loading mechanisms, generally illustrated in FIGS. 12A to 12E, may also be used alone, or in combination, as methods for lining the surface of the agent carrier or its cavities with hydrophilic or hydrophobic moieties prior to loading an agent, or with moieties that can conduct electric charges and/or participate in generating or propagating electric fields prior to loading an agent.

FIG. 12A provides an illustration of an embodiment of a method for charging an agent carrier with an agent. In this embodiment, the applicator tip 900 containing the agent carrier body 902 is connected to a hand-held applicator device (not shown) via its coupling rod 908. The agent carrier body 902 is at least partially immersed in a container 904 containing an agent 906. Ultrasonic vibration created by an ultrasonic transducer of the applicator device is coupled, via the coupling rod 908 to the applicator tip 900, and through it, to the agent carrier body 902. The vibration expels air from the micro channels and at least partially fills the micro channels and/or agent reservoirs within the agent carrier body 902 with agent 906.

FIG. 12B provides an illustration of another embodiment of a method for charging an agent carrier with an agent. In this embodiment, the agent carrier is a removable applicator tip 900′. The applicator tip 900′ and/or a separate agent carrier body 903 are at least partially immersed in a container 904′ containing an agent 906′. Ultrasonic vibration created by an external source 910 is applied to the container 904′, which expels air from the micro channels and/or agent reservoirs of the agent carrier contained in the applicator tip 900 (not shown) and/or the separated agent carrier body 903 and at least partially fills the micro channels and/or agent reservoirs of the agent carrier within the applicator tip 900′ and/or the separated agent carrier body 903 with agent 906′.

FIG. 12C provides an illustration of a vacuum chamber 912. Vacuum is applied at the port 914 to remove air from the chamber 912 and the air within the micro channels and/or agent reservoirs of an agent carrier held within an applicator tip 900′ or a separated agent carrier body 903′. When the vacuum is complete, a valve controlling the agent entry port 916 is opened so that agent stored in chamber 917 is drawn into the chamber 912 through the agent entry port 916 and into the micro channels and/or agent reservoirs in the agent carrier body 902″ in the applicator tip 900″ and/or the separated agent carrier body 903. Ingress of agent occurs via the pores in the tissue-contact surface of the agent carrier(s). Once charged with agent, the applicator tip 900″ and/or the separated agent carrier body 903′ is removed from the agent containing fluid and a seal layer may be applied over exposed surfaces.

FIG. 12D provides another embodiment of a method in which a vacuum is used to charge an agent carrier body 903′″ with agent 906″. Agent 906″ is held within a container 904′″. The agent carrier 903′″ is placed within the container 904′″ and at least partially submerged so that the pores of the tissue contact surface 920 of the agent carrier body 903″ are in the agent solution 906″. A vacuum is applied to port 918 to draw agent solution up through the micro channels in the agent carrier 903″ so that the micro channels and/or agent reservoirs are at least partially filled with the agent solution 906′″.

In an alternative embodiment of a method for charging an agent carrier body with agent, an agent can be directly injected into the port so that the air in the agent carrier (i.e. In the micro channels and/or agent reservoirs) is expelled and replaced by the agent.

FIG. 12E provides a similar method to that in FIG. 120 except an applicator tip 900″″ having an agent carrier body 902′″ is to be charged with agent. The applicator tip 900′″ is illustrated in cross section to illustrate that the applicator tip includes a reservoir 921 within its housing that is separate from any reservoir formed within the agent carrier body 902′. The applicator tip 900″ includes a vacuum port 922 that provides access to the reservoir 921. As above, a vacuum is applied at the vacuum port 922 which draws agent solution up through the micro channels in the agent carrier body 902″ so that the micro channels and/or agent reservoirs in either the agent carrier body 902′″ or applicator tip's 900″″ housing are at least partially filled with the agent solution 906″.

In an alternative embodiment of a method for charging an agent carrier or applicator tip having an agent carrier with agent, agent can be directly injected into a port so that the air in the agent carrier (e.g. In the micro channels and/or agent reservoirs) is expelled and replaced by the agent.

The agent carrier may be provided as either empty agent carriers or as charged agent carriers that are filled with an agent. Where empty agent carriers are provided, an end user will need to charge the agent carrier with agent prior to use.

The invention also relates to a method of charging the agent carrier with an agent and discharging agent from the agent carrier.

The method of discharging agent from the agent carrier or dispensing agent to a tissue surface includes applying the agent carrier to a tissue surface and dispensing agent from the agent carrier to the tissue surface. Preferably the process of dispensing the agent includes applying ultrasonic waves to the tissue surface to facilitate penetration of the agent into the tissue through sonophoresis.

As will be appreciated from the foregoing the agent carrier or an agent carrier body itself can be an item separable from the applicator device. In a preferred form the agent carrier or agent carrier body is a single use item that is removable or interchangeable. This aids in the sterility required for medical usage and facilitates among other things cleaning and sterilising of the hand-held applicator device between patients. The solid physical nature of the preferred embodiments facilitates mounting and handling of the agent carrier in circumstances where they are replaceable. Moreover, the use of a solid material for the agent carrier body to contain the agent facilitates loading of an agent into an agent carrier, packaging, handling of agent carrier bodies pre-loaded with agent. Importantly, the use of solid materials for the agent carrier body facilitate the propagation of ultrasonic waves that are used to move an agent through the agent carrier and enhances and/or permits the entry of an agent into the target tissue by sonophoresis.

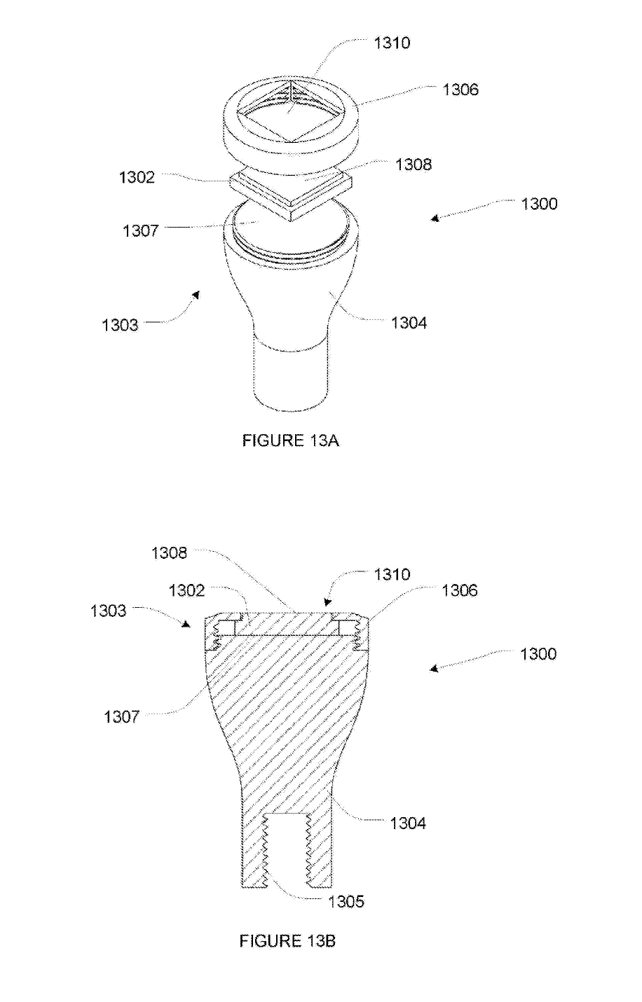

FIGS. 13A and 13B illustrate one embodiment agent carrier. The applicator tip 1300 is generally speaking equivalent to the agent carrier tip 102 shown in FIG. 1. In this example the agent carrier 1300 takes the form of an applicator tip with a removable and interchangeable agent carrier body.

The agent carrier 1300 includes the following main components: An agent carrier body 1302, and a tip housing 1303 that includes a tip body 1304 and an agent carrier body retaining cap 1306.

The agent carrier body 1302 is generally rectilinear in plan view, and in this example it is square. The agent carrier body 1302 may be made in accordance with any one of the examples given above or aspects described herein. The agent carrier body 1302 has a tissue contacting surface 1304.

The tip body 1304 serves to both connect the agent carrier 1300 to an agent applicator device and conduct transmission stimulus, in the form of ultrasonic energy to the agent carrier body 1302. To achieve this, the tip body 1304 is provided, on a first end thereof, with a mounting mechanism 1305 in the form of a screw thread. The mounting mechanism 1305 is used to make a mechanical connection with a corresponding connector of a handle assembly. The second end of the tip body 1304 is shaped to operate as a horn to conduct ultrasonic energy, via mating surface 1307, to the agent carrier body 1302.

The agent carrier body retaining cap 1306 serves to retain the agent carrier body 1302 and hold it in contact with the mating surface 1307. The agent carrier body retaining cap 1306 has an aperture 1310 formed in it, through which the tissue contacting surface 1308 of the agent carrier body 1302 is exposed in use. The agent carrier body retaining cap 1306 is mounted to the tip body 1304 using a screw thread.

As will be appreciated there are many morphological and mechanical variations can be made in such a system. For example the shape of the components, including the agent carrier body, and its associated tissue contacting surface may be varied. The present square embodiment is particularly convenient when the agent carrier body is made from a semiconductor material and its manufacturing process most conveniently outputs square components. The shape of the tip body can be varied to optimise transmission of ultrasonic energy if ultrasonic energy is used as a transportation stimulus. The shape of the aperture thorough which the tissue contacting surface of the agent carrier body is exposed can be varied. In some cases it may differ from the shape of the tissue contacting surface of the agent carrier body.

The method of engagement of the agent carrier retaining cap with the tip body can be varied widely to use any convenient type of mechanism. In this example engagement is by screw thread, however the agent carrier retaining cap could be press fit onto the tip body, or engaged with snap fasteners, or a bayonet fitting, to give a non-exhaustive list or alternatives. Similarly the mounting mechanism of the agent carrier body can be varied to use any known coupling mechanism.

An agent carrier having a plurality of agent carrier bodies, perhaps arranged in a pattern such as an array, could also be provided.

It will be understood that the invention disclosed and defined in this specification extends to all alternative combinations of two or more of the individual features mentioned or evident from the text or drawings. All of these different combinations constitute various alternative aspects of the invention.

1. An agent carrier for delivery of an agent to a tissue via a transportation stimulus, the agent carrier comprising:
an agent carrier body having a tissue-contacting surface for engaging tissues under treatment, the agent carrier body comprising a multiplicity of micro channels extending at least partially through the agent carrier body to the tissue-contacting surface configured for transportation of the agent to a tissue surface.

2. The agent carrier of claim 1, wherein the multiplicity of micro channels extending at least partially through the agent carrier body to the tissue-contacting surface are configured for retention of the agent within the agent carrier.

3. The agent carrier of claim 1, wherein the agent carrier is configured to conduct a transmission stimulus to transport the agent within the agent carrier and to the tissue surface.

4. The agent carrier of claim 1, wherein the agent carrier is configured to conduct a transmission stimulus to the tissue surface to permit penetration, enhance penetration, or permit and enhance penetration of the agent into the tissue.

5. The agent carrier of claim 1, wherein the agent carrier body is configured to conduct a transmission stimulus.

6. The agent carrier of claim 1, wherein the transportation stimulus is ultrasonic waves.

7. (canceled)

8. The agent carrier of claim 1, wherein the agent carrier body is made from one or more of a semi-conductor material, polymer, plastics material, or metal.

9. (canceled)

10. The agent carrier of claim 1, wherein the agent carrier body comprises a stack of layers, wherein the stack of layers comprises a tissue-contacting layer which comprises the tissue-contacting surface, and at least one other layer.

11.-14. (canceled)

15. The agent carrier of claim 1, wherein the tissue-contacting surface comprises micro_protrusions formed thereon, wherein the micro protrusions comprise a cavity defined by at least one of the micro channels.

16. The agent carrier of claim 1, wherein the agent carrier comprises an agent reservoir for storing the agent.

17. The agent carrier of claim 16, wherein the agent reservoir is at least partly formed within the agent carrier body.

18. (canceled)

19. The agent carrier of claim 1, wherein at least the micro channels are at least partly defined by internal exposed surfaces within the agent carrier body, wherein the internal exposed surfaces are configured to possess predetermined hydrophilic, hydrophobic, electro-conductive properties, or a combination thereof.

20. (canceled)

21. The agent carrier of claim 1, wherein the agent carrier comprises a housing, wherein the housing is configured to mechanically support the agent carrier body while the agent carrier body is in use.

22.-32. (canceled)

33. The agent carrier of claim 1, wherein the agent carrier is a consumable applicator tip configured for a one-time use as part of an applicator device.

34.-61. (canceled)

62. An agent carrier body for delivery of an agent into a tissue via a transportation stimulus, the agent carrier body comprising:
a tissue-contacting surface for engaging tissues under treatment, the agent carrier body comprising a multiplicity of micro channels extending at least partially through the agent carrier body to the tissue-contacting surface configured for transportation of the agent to a tissue surface.

63. The agent carrier body of claim 62, wherein the multiplicity of micro channels extending at least partially through the agent carrier body are configured for retention of the agent.

64. The agent carrier body of claim 62, wherein the agent carrier body is configured to conduct a transmission stimulus to facilitate at least one of:
transportation of the agent within the agent carrier body;
transportation of the agent to a tissue surface; and
penetration of the agent into the tissue.

65. The agent carrier body of claim 62, wherein the agent carrier body is made from one or more of a semi-conductor material, polymer, plastics material, or metal.

66.-72. (canceled)

73. The agent carrier body of claim 62, wherein the agent carrier body comprises an agent reservoir for storing the agent, wherein the agent reservoir is at least partly formed within the agent carrier body.

74. The agent carrier body of claim 62, wherein the micro channels, the agent reservoir, or both are defined at least partly by internal exposed surfaces within the agent carrier body, wherein the internal exposed surfaces are configured to possess predetermined hydrophilic, hydrophobic, electro-conductive properties, or a combination thereof.

75.-79. (canceled)

80. The agent carrier body of claim 62, wherein the agent carrier body is formed from silicon.

81.-124. (canceled)
